(19) United States
(12) Patent Application Publication
(10) Pub. No.: US 2026/0191742 A1
(43) Pub. Date: Jul. 9, 2026

(54) ENTERAL FEEDING TUBE FOR FEEDING PATIENTS SUCH AS NEWBORN BABIES, IN PARTICULAR PREMATURE BABIES

(71) Applicant: ASSISTANCE PUBLIQUE - HOPITAUX DE PARIS, Paris (FR)

(72) Inventor: Shivani SHANKAR-AGUILERA, Le Pecq (FR)

(21) Appl. No.: 19/134,066

(22) PCT Filed: Dec. 20, 2023
(86) PCT No.: PCT/EP2023/087150

§ 371 Date: May 29, 2025

(30) Foreign Application Priority Data
Dec. 21, 2022 (FR) FR2214158

(51) Int. Cl. A61J 15/00; A61M 1/00; A61M 25/00

(52) U.S. Cl. CPC A61J 15/0073; A61J 15/0003; A61J 15/0049; A61M 1/77; A61M 25/0032

(57) ABSTRACT

A tube for the enteral feeding of premature newborn babies including a first lumen having a proximal end and a distal end to be housed in the digestive tract, a second lumen having a proximal end and a distal end protruding from the distal end of this lumen, an inflatable/deflatable balloon, the balloon being positioned around a portion of the distal end, the tube including a third lumen having a distal end connected to the balloon and a proximal end that can be connected to inflation/deflation means, the tube being able to be inserted into the digestive tract, the distal end being housed in the stomach, the inflated balloon being able to be positioned in abutment against the cardia, so as to close the stomach, the distal end of the second lumen being positioned in the esophagus before the cardia.

### The present invention relates to a medical device for enteral feeding of a patient, notably an enteral feeding probe for feeding patients such as newborns, in particular premature infants.

In the field of neonatology, the number of so-called premature births that is, before 37 completed weeks of amenorrhea (WA) has risen, accounting for 11% of live births in 2010. Of these premature births, 85% are between 32 and 33 weeks of amenorrhea or between 34 and 36 weeks of amenorrhea, and 5% are very premature at less than 28 weeks of amenorrhea. In France, 60,000 newborns are born prematurely every year, of which 12,000 are born before 32 weeks of amenorrhea.

Neonatology is a relatively new specialty wherein advances in recent years have reduced the mortality and morbidity rate associated with prematurity.

The most essential needs of premature babies are:
respiratory: requiring respiratory assistance
nutritional: requiring the supply of enteral nutrition via a gastric tube or intravenous parenteral nutrition.

However, this type of respiratory or nutritional assistance requires management that can be invasive for the premature baby, generating discomfort. The progress made has led to major breakthroughs in less invasive and personalized care.

Thus at the respiratory level, in the case of intubation and ventilation, the aim is to limit the duration of this invasive mechanical procedure, favoring non-invasive ventilation as soon as possible by blowing air or O₂ into the nostrils at different pressures according to the needs of the newborn, in order to transmit it indirectly to the still immature lungs. Such a system is therefore less invasive but not airtight; as a result, there is more or less significant leakage of gas flow through the mouth outwardly but also to the stomach and the abdomen of the patient.

As a result, non-invasive ventilation presents side effects that are significant such as:
gastric and abdominal air distension, with air accumulating in the stomach and esophagus, known as CPAP (Continuous Positive Airway Pressure) Belly Syndrome, since at this age, the oropharyngeal structures and gastric sphincter are soft and loose structures;
poor digestive tolerance to milk administered by the gastric tube;
lung collapse due to abdominal distension and less efficient ventilation which requires increased ventilation pressures.

The result thereof is major discomfort for the newborn leading to frequent malaise and the start of a vicious cycle.

To remedy these shortcomings, feeding is reduced which is a poor solution to a non-problem since parenteral nutrition is then prolonged, for example by intravenous feeding which increases the risk of infection.

The ventilation level can also be lowered which leads to respiratory consequences, as respiratory assistance is then less, or even barely, effective. In order to limit the effects of a “bloated” stomach, it is also possible to empty the air manually by massage or by suction via the gastric tube, for example.

It is therefore important to be able to implement non-invasive ventilation safely while allowing safe enteral feeding using an enteral feeding tube.

Indeed, enteral nutrition is the most “natural” method of artificial nutrition as it uses a large part of the digestive system. Nutrients are thus delivered directly to the stomach or jejunum by various medical devices. Nutrients are then transformed in order to be absorbed in the intestine. Enteral nutrition is a treatment in its own right and requires rigorous hygiene.

Thus, a small tube is inserted through the nose or the mouth and descends directly into the stomach of the newborn patient. Small quantities of extracted breast milk or formula are then slowly poured into the stomach. If the baby responds well to this feeding, the dose is gradually increased. This method is therefore less invasive than a parenteral nutrition method.

Among the gastric probes used for enteral feeding, document WO-A-2018/009662 proposes a feeding tube comprising: a main tube, which can be fitted to the nose or in the mouth of a patient, the distal end of the main tube being intended to be positioned at the pylorus of the patient and has an inflatable balloon. This feeding device comprises a secondary tube which extends through the main tube and whose distal end extends beyond the distal end of the main tube into the jejunum. Once introduced into the stomach, the main tube is guided up to the pylorus, so that the secondary feeding tube projects into the jejunum. The balloon is then inflated to engage with the pylorus and close it. The main tube comprises suction orifices for aspirating the fluids present in the stomach into a suction tube.

A tube of this type thus enables safe feeding. However, this does not solve the problem of air entering the stomach when the premature baby is on non-invasive respiratory assistance. Furthermore, it does not encourage digestive activity in the stomach.

The purpose of the invention is therefore to propose a probe for enteral feeding of patients, notably premature newborns, comprising:
a first lumen constituting a feeding tube having a proximal end and a distal end intended to be housed in the digestive tract of the patient,
a second lumen having a proximal end and a distal end, the distal end of the feeding lumen projecting with respect to the distal end of this second lumen,
an inflatable and deflatable balloon, characterized in that the balloon is positioned around a part of the distal end of the feeding lumen,
the probe further comprising a third lumen having a distal end which is connected to the balloon and a proximal end which can be connected to means for inflating and deflating the balloon,
the probe being dimensioned to be inserted into the digestive tract of the patient so that the distal end of the feeding lumen is housed in the stomach, the balloon once inflated being able to be positioned in abutment against the cardia, in order to close the stomach while the distal end of the second lumen is positioned in the esophagus before the cardia.

Thus, advantageously, the feeding probe according to the invention makes it possible to feed the premature newborn by closing the stomach at the level of the cardia or gastric sphincter, which blocks the entry of air when the premature newborn is also under non-invasive ventilation. A lumen is understood to mean a duct or channel for the circulation of a fluid such as food or air.

Advantageously, the second lumen comprises at its distal end at least one orifice arranged in its cylindrical wall while its proximal end is connected to suction means, said second lumen constituting a tube for aspirating in the air contained in the esophagus.

Thus, the orifice(s) provided at the distal end of the second lumen aspirate the air contained in the esophagus, which thus also avoids esophageal distension associated with the accumulation of air or O₂ from non-invasive ventilation.

The feeding probe according to the invention avoids the gastric and abdominal distension related to the accumulation of air associated with non-invasive ventilation while enabling efficient feeding. In particular, this prevents the accumulation of air in the stomach from pushing back the diaphragm which could then act on the lungs by compressing them. The well-being and digestive and respiratory comfort of premature babies is thus improved, thus providing them with conditions that enhance their growth.

The feeding probe according to the invention can also be used for other pediatric or adult patients, for example with neuromuscular diseases, who, when fed enterally and under non-invasive ventilation, may suffer from gastric and abdominal distension, due to air accumulating in the stomach and esophagus.

According to a first embodiment, the medical device according to the invention therefore consists of a so-called tri-lumen (light) feeding probe consisting of three independent tubes defining the three lumens. Each tube is preferably a long and thin tube made of biocompatible material such as polyurethane, silicone, rubber or any other suitable material. The probe according to the invention is therefore simple to implement.

The first tube constituting a feeding tube has a proximal end and a distal end intended to be housed in the digestive tract of the patient, and is housed in a second tube having a proximal end and a distal end, the distal end of the feeding tube projecting from the distal end of this second tube. This second tube preferably forms the suction tube (lumen).

Advantageously, the third so-called inflation/deflation tube is also housed, preferably, in the second tube. The feeding tube and the third tube connected to the balloon thus extend adjacent over a large part of their length inside the second tube, however each proximal end of these tubes projects from the second tube to enable their respective connection to the various feeding and inflation/deflation means, while the proximal end of the second tube can be shaped to be connected to suction means.

The second tube or suction tube containing the other two tubes is for example a silicone tube whose distal end has at least one orifice, preferably five, in its cylindrical wall over a length of, for example, 5 cm, and whose proximal end can be connected to suction means such as a wall-mounted suction unit.

This tube houses the other two tubes. One of the tubes is the feeding tube and for newborn patients, silicone probes or tubes are generally used ranging in size from 6 to 14 Fr, preferably 6 Fr. The French unit (Fr) for diameters of this type of tube is 0.3 millimeters. The open distal end of this tube delivers food to the stomach. Additional orifices can also be provided in the cylindrical wall of this end of the feeding tube.

The third tube or inflation/deflation lumen allows air to be blown into the balloon and extends into the second tube along the feeding tube. A tube such as a pediatric silicone urinary catheter, preferably 6 Fr in size, can be used.

The inflatable and deflatable balloon is positioned around a part of the distal end of the feeding tube which protrudes from the second tube, while the third tube has a distal end which is connected to the balloon and a proximal end which is able to be connected to means for inflating and deflating the balloon.

The balloon consists of at least one flexible, for example tubular, wall positioned around the distal end part of the feeding tube which projects with respect to the second tube. The balloon, the ends of which are attached to the feeding tube at the distal end thereof, is thus positioned after the distal end of the second tube. The balloon can be inflated to close off the cardia. Preferably, the proximal end of the balloon can be shaped to lodge itself at the cardia without exerting too much pressure on the stomach, preferably with an end complementary in shape to the cardia.

According to a preferred embodiment of the invention, the balloon has a shape having at least two parts, the flexible wall being shaped to define a predominantly spherical part and an elongated proximal end part with a concave-shaped wall, extending as a continuation of the wall of the spherical part, when the balloon is inflated.

Preferably, the balloon wall also defines an elongated distal end part having a concave-shaped wall. The central part thus consists of a spherical segment, corresponding to the central part of a sphere and each end part has a concave-shaped wall. The shape of this balloon notably at the proximal end is thus complementary to the shape of the cardia and thus enables appropriate closure of the cardia.

According to a second embodiment, the medical device according to the invention consists of a so-called tri-lumen (light) feeding tube which consists of a single tube wherein are housed three ducts or channels forming the three lumens defining the three feeding, suction and inflation/deflation tubes. This tube is a long tube made of biocompatible material such as polyurethane, silicone, rubber or any other suitable material.

In this tube, the first duct forming the feeding lumen or tube projects with respect to the second duct forming the second suction lumen or tube, which is closed off, while the duct forming the inflation/deflation lumen or tube opens out at the balloon, mounted around the tube. The duct forming the feeding lumen opens into the stomach.

A subject of the invention is also a method for inserting a probe according to the invention into the digestive tract of a patient, wherein the distal end of the probe is inserted through the mouth or the nose of the patient, until the distal end of the feeding tube comprising the balloon is housed in the stomach, the balloon is inflated and the probe is pulled back until the balloon abuts against the cardia of the stomach and the probe is secured in position.

Advantageously, the probe according to the invention is used when the patient is under non-invasive ventilation, by feeding the patient through the feeding tube and by aspirating air from the esophagus through orifices provided at the distal end of the feeding tube, the proximal end of which is connected to suction means. Of course, such a tube can also be used when the patient is not under non-invasive ventilation.

To remove this tube, the balloon is deflated and the tube is removed.

The invention will now be described in greater detail with reference to the drawing wherein the figures represent:

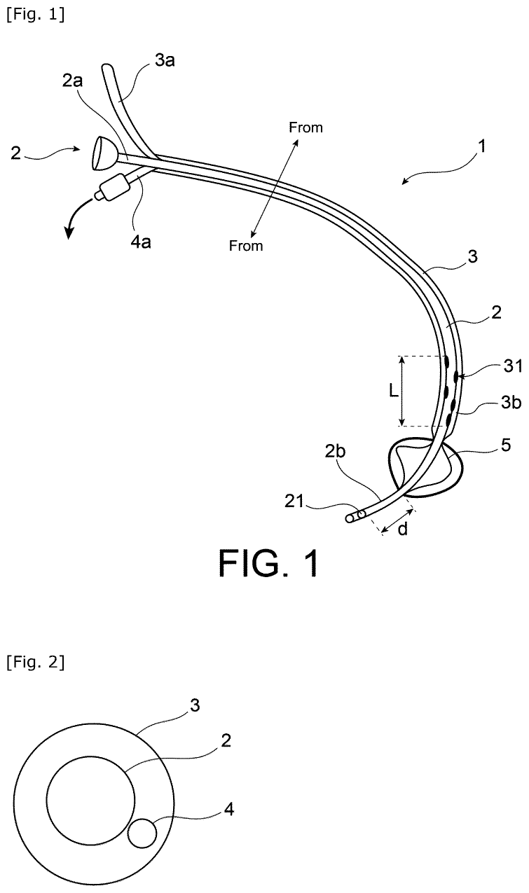

FIG. 1 an elevation view of a probe according to a first embodiment of the invention;

FIG. 2 a cross-sectional view of the probe of FIG. 1 along section A-A;

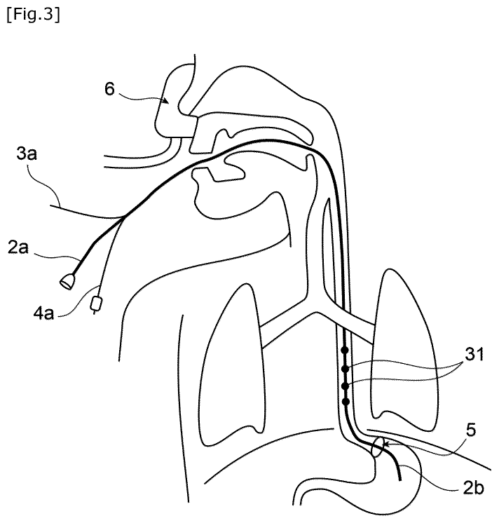

FIG. 3 a schematic cross-sectional view of a patient equipped with non-invasive ventilation and a probe according to the invention;

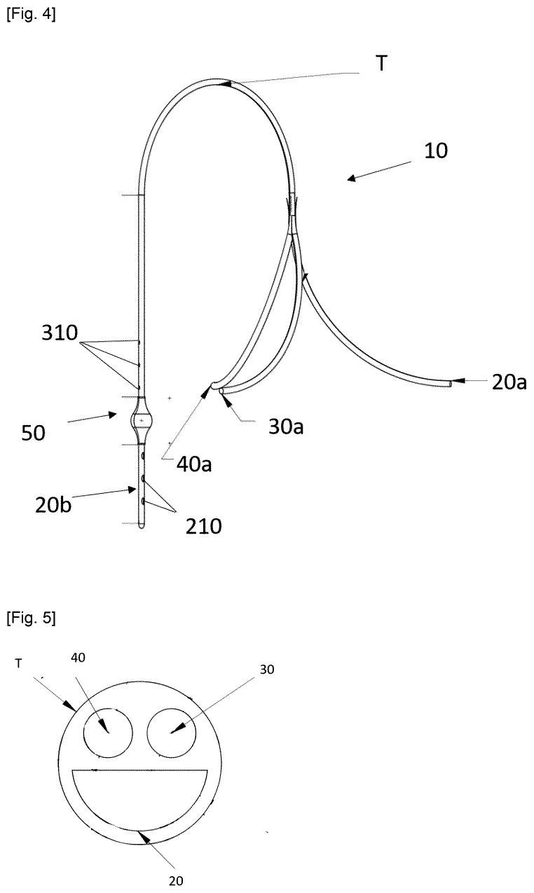

FIG. 4 an elevation view of a second embodiment of a probe according to the invention;

FIG. 5 a cross-sectional view of the probe of FIG. 4 along section B-B;

FIG. 6 a side perspective view of the balloon of FIG. 4; and

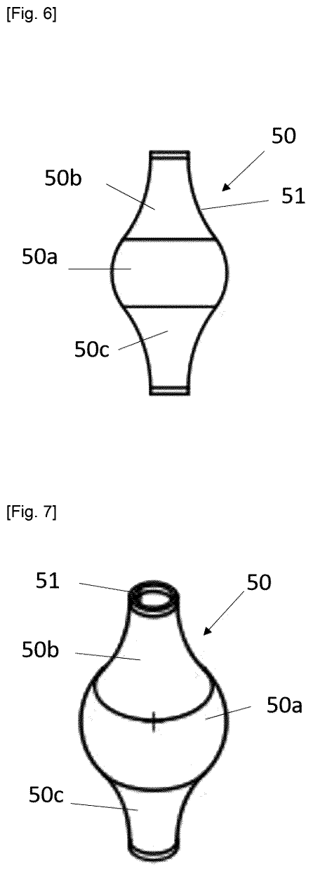

FIG. 7 a perspective top view of the balloon of FIG. 6.

An enteral feeding probe 1 according to the invention is intended to feed premature newborns in order to feed them while they are under non-invasive ventilation.

Non-invasive ventilation involves blowing air or oxygen into the lungs of the patient using a mask placed over the nose or nasal cannula.

The air thus ventilated not only enters the lungs but also reaches the digestive tract, which risks causing gastric distension.

A feeding probe 1 according to the invention is then used. This probe 1 according to a first embodiment consists of three tubes forming three lumens. The first tube 2 has a proximal end 2a and an opposite distal end 2b, the distal end 2b being the end for delivering food to the digestive tract while the proximal end 2a can be connected to food-containing means such as a sachet of breast milk or formula. This feeding tube 2 is housed in a second tube 3 forming a lumen of larger diameter, the feeding tube 2 extending through this second tube 3 and having a length greater than that of the second tube 3 so that the distal end 2b of the feeding tube 2 projects from the distal end 3b of the second tube 3.

The probe of the invention further comprises an inflatable and deflatable balloon 5. This balloon 5 consists of at least one tubular flexible wall positioned around the distal end part 2b of the feeding tube 2 which projects from the second tube 3, the balloon 5 whose ends are attached to the feeding tube 2 at the distal end 2b thereof, is thus positioned after the distal end 3b of said tube 3. The balloon 5 can thus be positioned at a distance d of approximately 4 cm from the tip of the distal end 2b of the feeding tube 2. Once inflated, the balloon 9 can then take on an elongated shape, for example an ovoid, spherical, elongated spherical, spherical with a conical end shape, etc.

A third tube 4 is housed in tube 3 (see FIG. 2), and has a distal end which is connected to the balloon 5 while its proximal end 4a is connected to means for inflating and deflating the balloon 5. The enclosure defined by the flexible wall of balloon 5 fills with air which inflates the balloon 5 (seen in FIG. 1 where the wall is shown in a rounded shape once the balloon is inflated (bold line)).

Tubes 2 and 4 extend for most of their length inside tube 3 but each proximal end 2a and 4a projects from tube 3 to allow their respective connection to the various feeding and inflation means, the proximal end 3a of tube 3 being shaped to be connected to the suction means.

Thus, tube 1 is inserted into the digestive tract of the patient. The length of feeding tube 2 is suitable for extending into the stomach of the patient while the length of second tube 3 is shorter and extends mainly into the esophagus. The distal end 2b may comprise additional orifices 21 in the cylindrical wall of this end of the feeding tube 2 to facilitate delivery of the food.

Once probe 1 has been inserted into the digestive tract, the distal end of the first tube 2 is located inside the stomach. The balloon 5 is then inflated. The probe 1 can then be pulled until the balloon 5 comes into abutment against the cardia and thus closes the stomach blocking the access of air from non-invasive ventilation. The balloon 5 therefore provides a seal between the stomach and the esophagus of the patient thus preventing the ingress of air from non-invasive ventilation and therefore abdominal distension.

The balloon 5 is then advantageously located between the distal end of the second tube 3, which extends into the esophagus and the distal end of the first tube 2 which is located in the stomach.

The distal end 3b of the second tube 3 comprises orifices 31 arranged in the cylindrical wall of said tube 3 while its proximal end 3a is connected to suction means. In this way, air from non-invasive ventilation located in the esophagus can also be aspirated through the orifices 31 in said tube 3 which then constitutes a suction tube. Five orifices 31 can be provided to extend over a length of L of 5 cm from the tip of the distal end 3b.

Thus, the probe 1 can be inserted into the stomach so that the distal end of the feeding tube 2 is housed in the stomach, the balloon 5, once inflated, can close the cardia of the stomach preventing the ingress of air from non-invasive ventilation 6 while the suction orifices 31 of the distal end 3b of the suction tube 3 are positioned in the esophagus and evacuate air from said esophagus (see FIG. 3).

FIG. 4 shows a second embodiment of the probe according to the invention. The probe 10 shown in FIG. 4 consists of a tube T comprising three lumens 20, 30, 40 formed by three ducts or channels arranged in the tube T. The first lumen 20 has a proximal end 20a and an opposite distal end 20b, the distal end 20b being the end opening into the digestive tract and enabling the delivery of food into the digestive tract while the proximal end 20a can be connected to food-containing means such as a sachet of breast milk or formula. The distal end 20b may comprise additional orifices 210 in the cylindrical wall of this end to facilitate delivery of the food.

In this tube T, the first duct forming the feeding lumen or tube 20 projects with respect to the second duct forming the second suction lumen or tube 30, which is closed off while the duct forming the inflation/deflation lumen or tube 40 opens out at the balloon, mounted around the tube T. The duct forming the feeding lumen opens into the stomach.

The second lumen (channel) 30 thus comprises at its distal end orifices 310 arranged in the cylindrical wall of said lumen 30, extending above a balloon 50 positioned around the distal end of the tube T and intended to close the stomach, while its proximal end 30a is connected to suction means. In this manner, the air from non-invasive ventilation and located in the esophagus can also be aspirated through the orifices 310 in said lumen 30 which then constitutes a suction tube. Up to five orifices 310 can be provided extending over a length of L of 5 cm from the tip of the distal end.

The third lumen 40 has a distal end which is connected to the balloon 50 while its proximal end 40a is connected to means for inflating and deflating the balloon 50.

The proximal ends 20a, 30a, 40a of the three lumens 20, 30, 40 preferably comprise endpieces for connection to the various feeding, suction and inflation/deflation means.

FIGS. 6 and 7 show an alternative balloon 50 which comprises at least one flexible wall 51 intended to be positioned around part of the distal end of tube T or tube 2 in the first embodiment. This wall 51 is shaped to define a predominantly spherical shape 50a and an elongated proximal end part having a concave-shaped wall 50b, extending as a continuation of the wall of the spherical part 50a, when the balloon 50 is inflated. Preferably, the wall 51 of the balloon 50 defines an elongated distal end part having a concave-shaped wall 50c.

Such a shape allows an advantageous adaptation to the cardia in order to close the stomach.

1. A probe for enteral feeding of a patient, notably premature newborns, comprising:
a first lumen constituting a feeding tube having a proximal end and a distal end intended to be housed in a digestive tract of the patient;
a second lumen having a proximal end and a distal end, the distal end of the feeding lumen projecting with respect to the distal end of the second lumen; and
an inflatable and deflatable balloon, the balloon is positioned around a part of the distal end of the feeding lumen;
the probe further comprising a third lumen having a distal end which is connected to the balloon and a proximal end which can be connected to means for inflating and deflating the balloon, constituting an inflation/deflation tube for the balloon; and
the probe being dimensioned to be inserted into the digestive tract of the patient so that the distal end of the feeding lumen is housed in the stomach, the balloon once inflated being able to be positioned in abutment against a cardia, to close a stomach while the distal end of the second lumen is positioned in an esophagus before the cardia.

2. The probe according to claim 1, characterized in that the second lumen comprises at the second lumen distal end at least one orifice arranged in a cylindrical wall while the second lumen proximal end can be connected to suction means, said second lumen constituting a tube for aspirating air contained in the esophagus.

3. The probe according to claim 1, characterized in that the three lumens consist of three independent tubes,
a first tube constituting the feeding tube having a proximal end and a distal end intended to be housed in the digestive tract of the patient;
the feeding tube being housed in a second tube having a proximal end and a distal end, the distal end of the feeding tube projecting from the distal end of the second tube, the second tube constituting the tube for aspirating—the air contained in the esophagus; and
the third tube having a distal end which is connected to the balloon and a proximal end which can be connected to the means for inflating and deflating the balloon,
the inflatable and deflatable balloon being positioned around a part of the distal end of the feeding tube which projects from the second tube.

4. The probe according to claim 3, characterized in that the third tube is housed in the second tube.

5. The probe according to claim 3, characterized in that each tube is a long and thin tube made of biocompatible material such as polyurethane, silicone, rubber or any other suitable material.

6. The probe according to claim 1, characterized in that the probe consists of a single tube wherein three ducts are arranged forming the three lumens.

7. The probe according to claim 6, characterized in that, in the tube, the lumen forming the feeding tube projects with respect to the lumen forming the second suction tube, the latter being closed off while the lumen forming the third tube opens out at the balloon, mounted around the tube.

8. The probe according to claim 1, characterized in that the balloon comprises at least one tubular flexible wall positioned around a part of the distal end of the feeding tube which projects from the second tube.

9. The probe according to claim 8, characterized in that the proximal end of the balloon is shaped to lodge itself at the cardia.

10. The probe according to claim 1, characterized in that the balloon comprises at least one flexible wall positioned around a part of the distal end of the feeding tube which protrudes from the second tube, said wall defining a spherical shape and an elongated proximal end part having a concave-shaped wall.

11. The probe according to claim 10, characterized in that the wall of the balloon defines an elongated distal end part having a concave-shaped wall.
